(19) United States
(12) Patent Application Publication
(10) Pub. No.: US 2008/0032184 A1
(43) Pub. Date: Feb. 7, 2008

(54) Stable Trifluorostyrene Containing Compounds, And Their Use In Polymer Electroyte Membranes

(76) Inventors: Zhen-yu Yang, Hockessin, DE (US); Mark Roelofs, Hockessin, DE (US)

Correspondence Address: Gorman , Thomas W;E.I Du pont de nemours and company, 4417 Lancaster Pike, Wilmington, DE, 19805, US

(21) Appl. No.: 11/547,583

(22) PCT Filed: Jun. 25, 2004
(86) PCT No.: PCT/US04/20702

§ 371 Date: Jul. 2, 2007

Related U.S. Application Data
(60) Provisional application No. 60/568,960, filed on May 7, 2004.

(51) Int. Cl. C07C 317/14; H01M 8/00

(52) U.S. Cl. 429/46; 568/34; 568/35

(57) ABSTRACT

A monomer comprising the structure (1a) or (1b) wherein Z comprises S, SO₂, or POR wherein R comprises a linear or branched perfluoroalkyl group of 1 to 14 carbon atoms optionally containing oxygen or chlorine, an aryl or substituted aryl group of 6 to 12 carbon atoms, or an alkyl of 1 to 8 carbon atoms; R_{F}comprises a linear or branched perfluoroalkene group of 1 to 20 carbon atoms, optionally containing oxygen or chlorine; Q is chosen from F, —OM, NH2, —N(M)SO₂R²_{F}, and —C(M)(SO₂R²F)₂, wherein M comprises H, an alkali cation, or ammonium; and R²F groups comprise perfluorinated or partially fluorinated alkyl, and may optionally include ether oxygens; and n is 1 or 2 for (1a), and n is 1, 2, or 3 for (1b). These monomers are used in the preparation of homopolymers and copolymers that are useful in preparing polymer electrolyte membranes. Electrochemical cells, such as fuel cells, containing these membranes are also described.

### FIELD OF THE INVENTION

The present invention relates to a novel compound and its use in electrochemical cells as an electrolyte, and more particularly to the use of the compound as an electrolyte in fuel cells. This invention was made with government support under Contract No. DE-FC04-02AL67606 awarded by the U.S. Department of Energy. The government has certain rights in the invention.

### BACKGROUND OF THE INVENTION

Electrochemical cells, such as fuel cells and lithium-ion batteries are known. Depending on the operating conditions, each type of cell places a particular set of requirements upon the electrolytes used in them. For fuel cells, this is typically dictated by the type of fuel, such as hydrogen or methanol, used to power the cell and the composition of the membrane used to separate the electrodes. Proton-exchange membrane fuel cells, powered by hydrogen as the fuel, could be run at higher operating temperatures than currently employed to take advantage of lower purity feed streams, improved electrode kinetics, better heat transfer from the fuel cell stack to improve its cooling. Waste heat is also employed in a useful fashion. However, if current fuel cells are to be operated at greater than 100° C. then they must be pressurized to maintain adequate hydration of typical proton-exchange membranes, such DuPont Nafion® perfluorosulfonic acid membrane, to support useful levels of proton conductivity.

There is an ongoing need to discover novel electrolytes that improve the performance of the latest generation of electrochemical cells, such as fuel cells and lithium-ion batteries, to develop new membrane materials that will maintain adequate proton conductivity at lower levels of hydration.

### SUMMARY OF THE INVENTION

In a first aspect, the invention provides a monomer comprising the structure 1a or 1b:

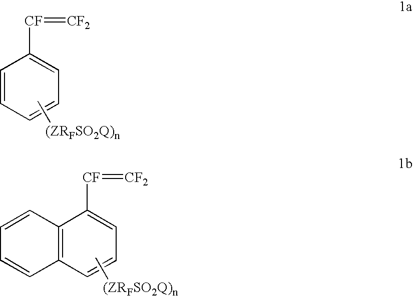

wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine; Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms, wherein the alkyl or aryl groups may be perfluorinated or partially fluorinated; and

n is 1 or 2 for 1a, and n is 1, 2, or 3 for 1b.

In a second aspect, the invention provides a homopolymer comprising the structure 2a or 2b:

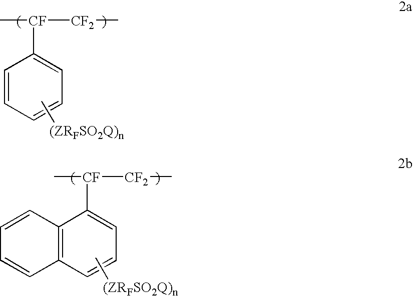

wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and

n is 1 or 2 for 2a, and n is 1, 2, or 3 for 2b.

In a third aspect, the invention provides a polymer chosen from:

(b) copolymers comprising structure 3a or 3b:

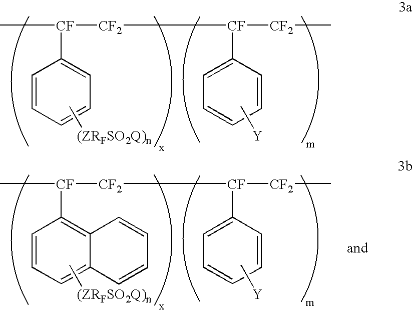

(c) cross linked copolymers comprising structure 4a or 4b:

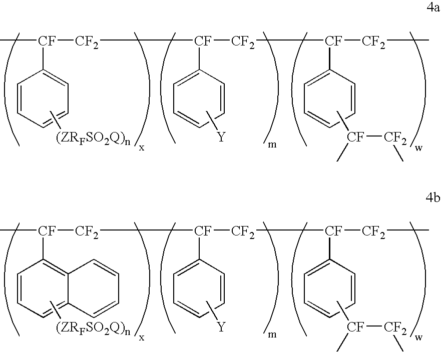

wherein in 3a, 3b, 4a or 4b:

Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;

Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;

n is 1 or 2 for 3a and 4a, and n is 1, 2, or 3 for 3b or 4b; and

x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1 .

In a fourth aspect, the invention provides a polymer electrolyte membrane prepared from a homopolymer or copolymer chosen from:

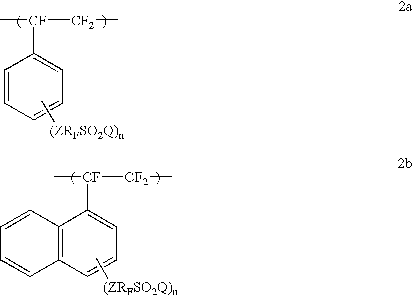

wherein Z comprises S, SO2, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
RF comprises a linear or branched perfluoroalkene group of 1 to 20 carbon atoms, optionally containing oxygen or chlorine; Q is chosen from F, —OM, —NH2, —N(M)SO2R2F, and —C(M)(SO2R2F)2, wherein M comprises H, an alkali cation, or ammonium, and R2F groups comprise alkyl of 1 to 14 carbon atoms that may optionally include ether oxygens or aryl of 6 to 12 carbon atoms wherein the alkyl or aryl groups may be perfluorinated or partially fluorinated; and

n is 1 or 2 for 2a, and n is 1, 2, or 3 for 2b

(a) homopolymers comprising the structure 2a or 2b:

(b) copolymers comprising structure 3a or 3b:

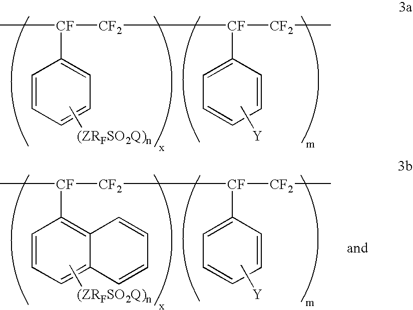

(c) cross linked copolymers comprising structure 4a or 4b:

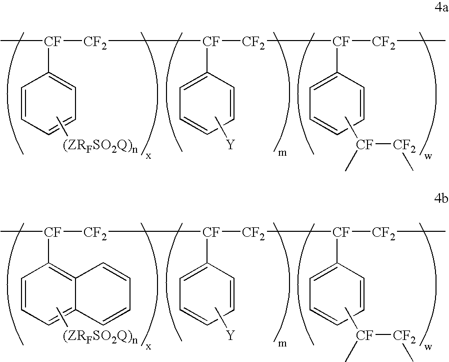

wherein in 3a, 3b, 4a or 4b:

Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;

Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;

n is 1 or 2 for 3a and 4a, and n is 1, 2, or 3 for 3b or 4b; and

x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

In the fifth aspect, the invention provides a catalyst coated membrane comprising a polymer electrolyte membrane chosen from:

(a) a membrane prepared from copolymers comprising the structure 2a or 2b:

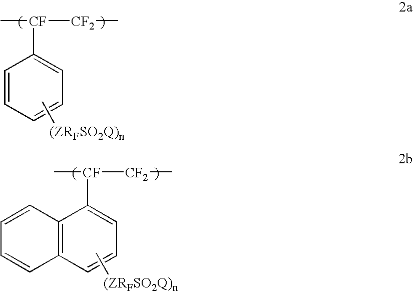

wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²F, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and

n is 1 or 2 for 2a, and n is 1, 2, or 3 for 2b,

(b) copolymers comprising structure 3a or 3b:

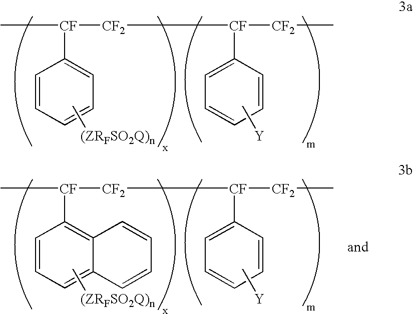

(c) cross linked copolymers comprising structure 4a or 4b:

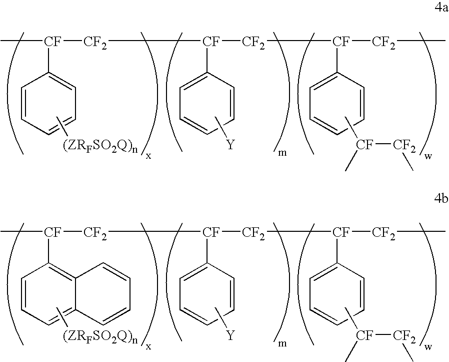

wherein in 3a, 3b, 4a or 4b:

Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;

Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;

n is 1 or 2 for 3a and 4a, and n is 1, 2, or 3 for 3b or 4b; and x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

In a sixth aspect, the invention provides a membrane electrode assembly comprising a polymer electrolyte membrane, having a first surface and a second surface, wherein the membrane is prepared from a homopolymer or copolymer chosen from:

(a) a homopolymer having the structure 2a or 2b:

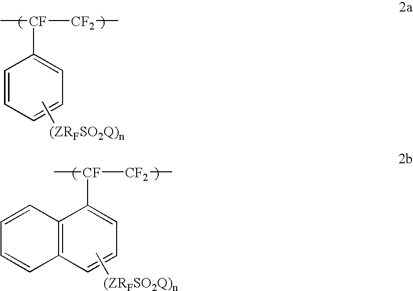

wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and

n is 1 or 2 for 2a, and n is 1, 2, or 3 for 2b

(b) copolymers comprising structure 3a or 3b:

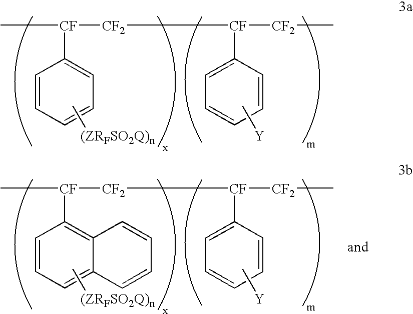

(c) cross linked copolymers comprising structure 4a or 4b:

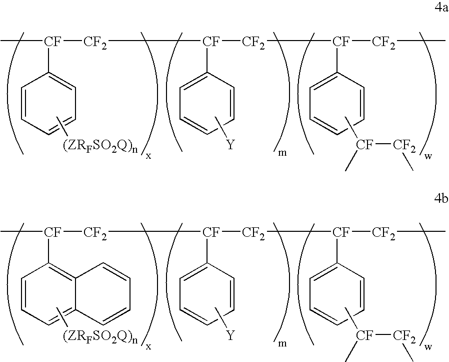

wherein in 3a, 3b, 4a or 4b:

Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;

Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;

n is 1 or 2 for 3a and 4a, and n is 1, 2, or 3 for 3b or 4b; and x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

In the sixth aspect, the membrane electrode assembly comprises a polymer electrolyte membrane further comprising a porous support. In the sixth aspect, the membrane electrode assembly further comprises at least one electrode prepared from an electrocatalyst coating composition present on the first and second surfaces of the membrane. It also further comprises at least one gas diffusion backing. Alternately, the membrane electrode assembly further comprises a gas diffusion electrode present on the first and second surfaces of the membrane, wherein the gas diffusion electrode comprises a gas diffusion backing and an electrode prepared from an electrocatalyst containing composition.

In a seventh aspect, the invention provides an electrochemical cell, such as a fuel cell, comprising a membrane electrode assembly, wherein the membrane electrode assembly comprises a polymer electrolyte membrane, having a first surface and a second surface, wherein the membrane is prepared from a homopolymer or copolymer chosen from:

(a) a homopolymer having the structure 2a or 2b:

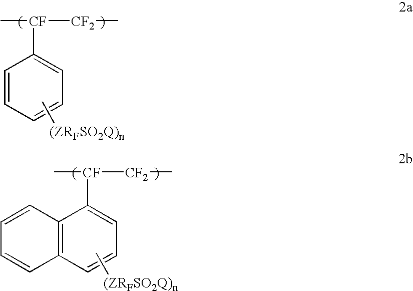

wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and

n is 1 or 2 for 2a, and n is 1, 2, or 3 for 2b

(b) copolymers comprising structure 3a or 3b:

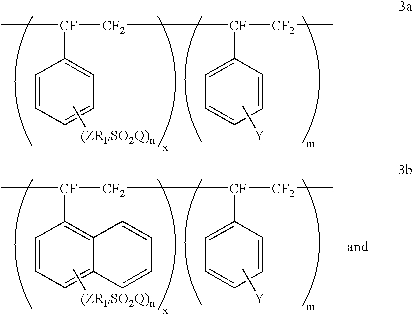

(c) cross linked copolymers comprising structure 4a or 4b:

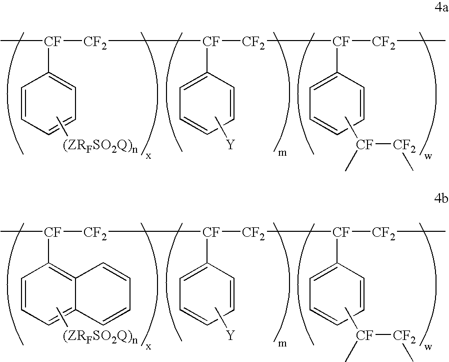

wherein in 3a, 3b, 4a or 4b:

Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;

Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;

n is 1 or 2 for 3a and 4a, and n is 1, 2, or 3 for 3b or 4b; and x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

In the seventh aspect, the invention provides a fuel cell comprising a polymer electrolyte membrane further comprising a porous support.

In the seventh aspect, the fuel cell further comprises at least one electrode prepared from an electrocatalyst containing composition, e.g., an anode and a cathode, present on the first and second surfaces of the polymer electrolyte membrane. It also further comprises at least one gas diffusion backing. Alternately, the membrane electrode assembly further comprises a gas diffusion electrode present on the first and second surfaces of the membrane, wherein the gas diffusion electrode comprises a gas diffusion backing and an electrode prepared from an electrocatalyst containing composition.

In the seventh aspect, the fuel cell further comprises a means for delivering a fuel to the anode, a means for delivering oxygen to the cathode, a means for connecting the anode and cathode to an external electrical load, hydrogen or methanol in the liquid or gaseous state in contact with the anode, and oxygen in contact with the cathode. The fuel is in the liquid or vapor phase. Some suitable fuels include hydrogen, alcohols such as methanol and ethanol; ethers such as diethyl ether, etc.

### BRIEF DESCRIPTION OF DRAWINGS

FIG. 1 is a schematic illustration of a single cell assembly.

FIG. 2 is a schematic illustration of the lower fixture of a four-electrode cell for in-plane conductivity measurement.

### DETAILED DESCRIPTION OF THE INVENTION

The monomers of the invention that are small molecules may be used to prepare homopolymers or copolymers that are useful as electrolytes in the preparation of the solid polymer electrolyte membranes. These polymer electrolyte membranes are used to make catalyst coated membranes that are a component of fuel cells. These homopolymers or copolymers are also useful as electrolytes in other electrochemical cells, such as batteries, chloralkali cells, electrolysis cells, sensors, electrochemical capacitors, and modified electrodes.

### Monomer:

The monomers of the invention have the following structure:

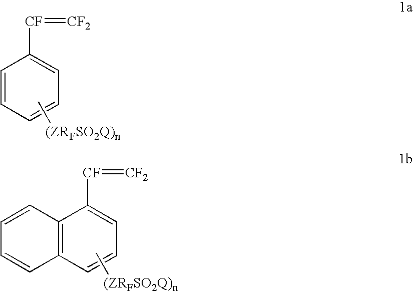

wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine; Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms, wherein the alkyl or aryl groups may be perfluorinated or partially fluorinated; and

n is 1 or 2 for 1a, and n is 1, 2, or 3 for 1b.

Some suitable perfluorinated alkylene groups R_{F} may comprise (CF₂)_{q} wherein q=1 to 16, (CF₂)_{q}OCF₂CF₂ wherein q=1 to 12, or (CF₂CF(CF₃)O)_{q}CF₂CF₂ wherein q=1 to 6. Typically R_{F} may comprise (CF₂)_{q} wherein q=1 to 4; (CF₂)_{q}OCF₂CF₂ wherein q=1 to 4; or (CF₂CF(CF₃)O)_{q}CF₂CF₂ wherein q=1 to 2. R²_{F} groups typically are chosen from methyl, ethyl, propyl, butyl, and phenyl, each of which may be partially fluorinated or perfluorinated. More typically R²_{F} groups are chosen from perfluoromethyl, perfluoroethyl, and perfluorophenyl. Typically the group Z linking R_{F} with the trifluorostyrene ring is S (sulfide linkage) or SO₂ (sulfone linkage). Typically n is 1.

### A. Synthesis of Monomers:

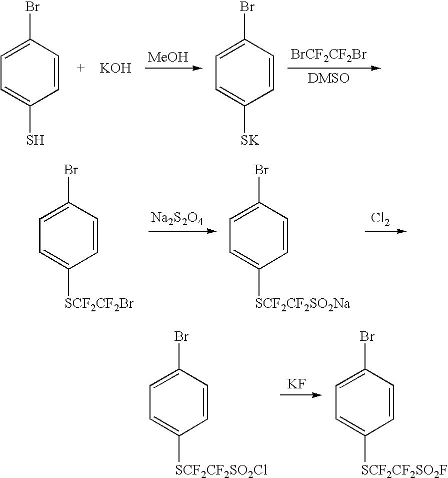

BrC₆H₄SH was converted into the potassium salt by reacting with KOH in MeOH. After being dried in vacuum, the salt reacted with BrCF₂CF₂Br in DMSO to give BrC₆H₄SCF₂CF₂Br in high yield. Sulfination with Na₂S₂O₄ and then chlorination produced the corresponding fluorosulfonyl chloride, followed by fluorine-chlorine exchange to give the fluorosulfonyl fluoride BrC₆H₄SCF₂CF₂SO₂F.

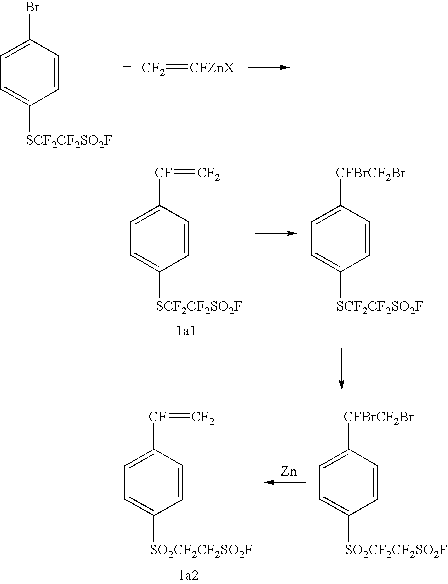

Coupling reaction of BrC₆H₄SCF₂CF₂SO₂F with CF₂=CFZnX with Pd catalysis according to Burton's method (Burton et al, JOC 53, 2714, 1988) gave the coupled product 1a1, which could be protected with Br₂ and then oxidized to form the corresponding sulfone. Debromination of the sulfone with Zn gave monomer 1a2.

Other monomers such as trifluorostyrene and 1,4-bis(trifluorostyrene) were made in similar fashion according to Burton's method. Alternatively, the monomers can be prepared by reaction of sodium or potassium salts of bromothiophenol with IRFSO₂E, wherein E is a protecting group such as imidazol. The resulting product BrC₆H₄SRFSO₂E undergoes a palladium catalyzed coupling reaction with CF₂═CFZnX to give the CF₂═CFC₆H₄SRFSO₂E

Homopolymers and Copolymers:

These monomers are used to prepare homopolymers and copolymers using the following procedure: Homo- and copolymerization of 1 may be conducted by neat polymerization, solution polymerization, suspension polymerization, or emulsion polymerization. Typical initiators such as Lupersol® 11 and perfluoroacyl peroxide were used in suspension polymerization or solution polymerization. In an aqueous polymerization, inorganic peroxides such as potassium persulfates (KPS) and ammonium persulfate (APS) obtained from Aldrich, Milwaukee, Wis.) were used as an initiator, or fluorinated organic salts such as ammonium perfluorooctanoate and fluorinated alkane sulfonates, or non-fluorinated surfactants such as dodecylaminie hydrochloride salt were used as surfactants. Monomers represented by structure 1 were typically used in aqueous emulsion polymerization. Molecular weight of polymers can be controlled by addition of chain transfer agents such as halocarbons, CHCl₃, fluorinated iodides and bromides, MeOH, ethers esters and alkanes. Polymers were isolated by coagulation. The polymers have high thermal stability and may be pressed into thin films. Some of the polymers may also be dissolved in certain solvents such as trifluorotoluene and 2,5-dichlorotrifluorotoluene. Thin films may also be cast from these polymer solutions. Slightly cross linked polymers such as those having the structure 4 have improved mechanical properties and reduced excess water uptake.

The resulting homopolymer formed by the above procedure has the following structure:

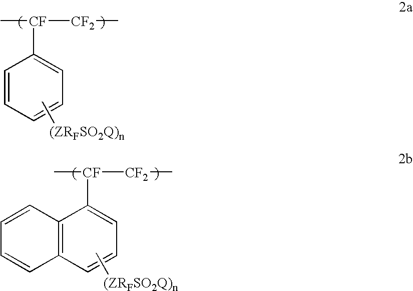

wherein Z comprises S, SO_{2,} or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and

n is 1 or 2 for 2a, and n is 1, 2, or 3 for 2b.

Some suitable perfluorinated alkylene groups R_{F} may comprise (CF₂)_{q} wherein q=1 to 16, (CF₂)_{q}OCF₂CF₂ wherein q=1 to 12, or (CF₂CF(CF₃)O)_{q}CF₂CF₂ wherein q=1 to 6. Typically R_{F} may comprise (CF₂)_{q} wherein q=1 to 4; (CF₂)_{q}OCF₂CF₂ wherein q=1 to 4; or (CF₂CF(CF₃)O)_{q}CF₂CF₂ wherein q=1 to 2. R²_{F} groups typically are chosen from methyl, ethyl, propyl, butyl, and phenyl, each of which may be partially fluorinated or perfluorinated. More typically R²_{F} groups are chosen from perfluoromethyl, perfluoroethyl, and perfluorophenyl. Typically the group Z linking R_{F} with the trifluorostyrene ring is S (sulfide linkage) or SO₂ (sulfone linkage). Typically n is 1.

The resulting copolymer formed using the above procedure are represented by the structures chosen from (b) copolymers comprising structure 3a or 3b:

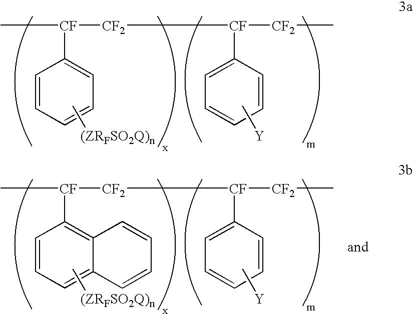

(c) cross linked copolymers comprising structure 4a or 4b:

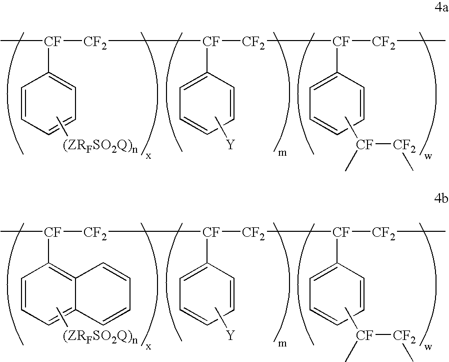

wherein in 3a, 3b, 4a or 4b:

Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;

R_{F} comprises a linear or branched perfluoroalkene group of 1 to 20 carbon atoms, optionally containing oxygen or chlorine;

Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;

Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;

n is 1 or 2 for 3a and 4a, and n is 1, 2, or 3 for 3b or 4b; and

x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1. Suitable perfluorinated alkylene groups R_{F} may comprise (CF₂)_{q} wherein q=1 to 16, (CF₂)_{q}OCF₂CF₂ wherein q=1 to 12, or (CF₂CF(CF₃)O)_{q}CF₂CF₂ wherein q=1 to 6. Typically R_{F} may comprise (CF₂)_{q} wherein q=1 to 4; (CF₂)_{q}OCF₂CF₂ wherein q=1 to 4; or (CF₂CF(CF₃)O)_{q}CF₂CF₂ wherein q=1 to 2. R²_{F} groups typically are chosen from methyl, ethyl, propyl, butyl, and phenyl, each of which may be partially fluorinated or perfluorinated. More typically R²_{F} groups are chosen from perfluoromethyl, perfluoroethyl, and perfluorophenyl. Typically the group Z linking R_{F} with the trifluorostyrene ring is S (sulfide linkage) or SO₂ (sulfone linkage). Typically n is 1. Some suitable substituents Y are chosen from hydrogen, chlorine, fluorine, methyl, ethyl, methoxy, perfluoromethyl, perfluoroethyl, perfluorobutyl, perfluoromethoxy, and —CF₂CF(CF₃)OCF₂CF₃. Typically, m and x and w are mole fractions, wherein m is 0.1 to 0.4; and x is 0.9 to 0.6 in structure 3a or 3b, and wherein m is 0.2 to 0.6; x is 0.4 to 0.8; and w is 0.002 to 0.01 in structure 4a or 4b.

### Membrane:

The hompolymer and copolymers can be cast into thin films from their solutions. Typically, tetrahydrofuran, trifluorotoluene and mixtures thereof were used as solvents. The cast films were transparent and flexible. The films also may also be made by thermally pressing at 200 to 250° C. The films may be hydrolyzed with bases such as MOH, M₂CO₃ wherein M=Li⁺, Na⁺, K⁺ or Cs⁺, or MOH in a mixture of MeOH, DMSO and water. The hydrolysis is typically carried out at room temperature to 150° C., more typically at room temperature to 80° C. Treatment of polymeric salts with acids such as HNO₃ gave polymeric acids. It has been found that the particular linkage groups Z used here give rise to increased thermal stability of the ion exchange polymers that are in acid form. Polymers represented by structures 2, 3 and 4 with Q=F may be converted to the corresponding sulfonimide by reaction with CF₃SO₂NH₂ and base. Polymers represented by structures 2, 3 and 4 with Q=NH₂ may be converted to the corresponding sulfonimide by reaction with R²_{F}SO₂F and base.

The sulfide homopolymers (Z=S) 2a and 2b or copolymers 3a and 3b may be oxidized to sulfone polymers (Z═SO₂) using CrO₃ or hydrogen peroxide.

The ionomers of homopolymers and copolymers identified above may be imbibed into a porous support to form a polymer electrolyte membrane having improved mechanical properties and dimensional stability. These membranes are capable of operating at a temperature of above 100° C. Ionomers may have 5% to 99.9% of membrane weight, typically, 20 to 98%, more typically 50 to 90%.

### Porous Support:

The porous support of the membrane may be made from a wide range of components. The porous support of the present invention may be made from a hydrocarbon such as a polyolefin, e.g., polyethylene, polypropylene, polybutylene, copolymers of those materials, and the like. Perhalogenated polymers such as polychlorotrifluoroethylene may also be used. For resistance to thermal and chemical degradation, the support preferably is made of a highly fluorinated polymer, most preferably perfluorinated polymer.

For example, the polymer for the porous support can be a microporous film of polytetrafluoroethylene (PTFE) or a copolymer of tetrafluoroethylene with other perfluoroalkyl olefins or with perfluorovinyl ethers. Microporous PTFE films and sheeting are known which are suitable for use as a support layer. For example, U.S. Pat. No. 3,664,915 discloses uniaxially stretched film having at least 40% voids. U.S. Pat. Nos. 3,953,566; 3,962,153 and 4,187,390 disclose porous PTFE films having at least 70% voids.

Alternatively, the porous support may be a fabric made from fibers of the support polymers discussed above woven using various weaves such as the plain weave, basket weave, leno weave, or others. A membrane suitable for the practice of the invention can be made by coating the porous support fabric with the compound of the invention to form a composite membrane. To be effective the coating must be on both the outside surfaces as well as distributed through the internal pores of the support. This may be accomplished by impregnating the porous support with a solution or dispersion of the polymer suitable for the practice of the invention using a solvent that is not harmful to the polymer or the support, and under impregnation conditions that can form a thin, even coating of the polymer on the support. The support with the solution/dispersion is dried to form the membrane. If desired, thin films of the ion exchange polymer can be laminated to one or both sides of the impregnated porous support to prevent bulk flow through the membrane that can occur if large pores remain in the membrane after impregnation.

It is preferred for the ion exchange polymer to be present as a continuous phase within the membrane.

Other forms of the solid polymer electrolyte membrane include the PTFE yarn embedded type and the PTFE fibril dispersed type, wherein the PTFE fibril is dispersed in the ion exchange resin as disclosed in 2000 Fuel Cell Seminar (10/30 to 11/2, 2000, Portland, Oreg.) Abstracts, p-23.

### Electrochemical Cell:

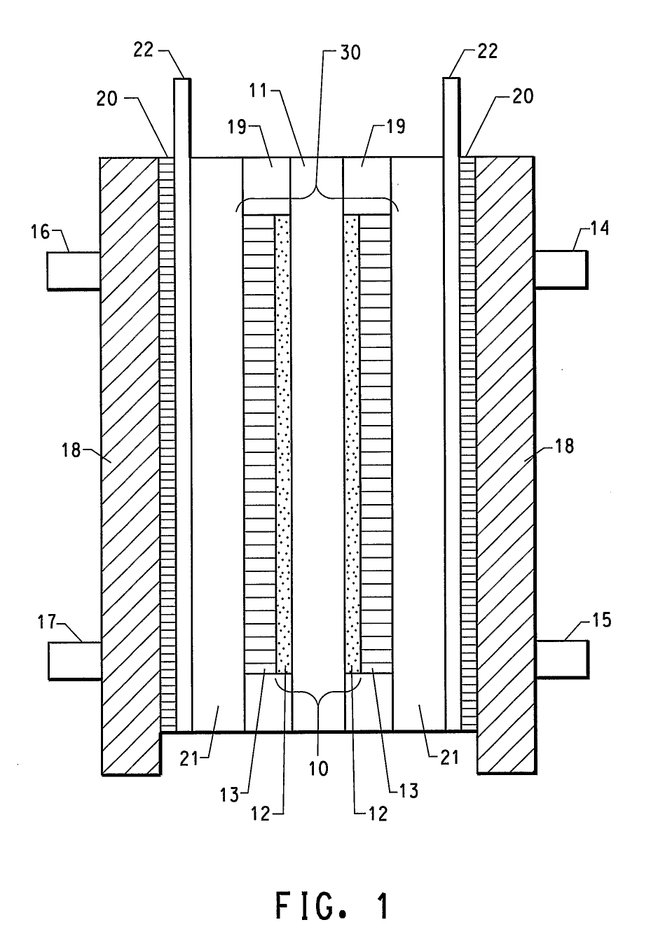

As shown in FIG. 1, an electrochemical cell, such as a fuel cell, comprises a catalyst coated membrane (CCM) (10) in combination with at least one gas diffusion backing (GDB) (13) to form an unconsolidated membrane electrode assembly (MEA). The catalyst coated membrane (10) comprises an ion exchange polymer membrane (11) discussed above and catalyst layers or electrodes (12) formed from a electrocatalyst coating composition. The fuel cell is further provided with an inlet (14) for fuel, such as liquid or gaseous alcohols, e.g. methanol and ethanol; or ethers such as diethyl ether, etc., an anode outlet (15) a cathode gas inlet (16) a cathode gas outlet (17) aluminum end blocks (18) tied together with tie rods (not shown), a gasket for sealing (19) an electrically insulating layer (20) and graphite current collector blocks with flow fields for gas distribution (21) and gold plated current collectors (22).

The fuel cell utilizes a fuel source that may be in the liquid or gaseous phase, and may comprise hydrogen, an alcohol or ether. Typically a methanol/water solution is supplied to the anode compartment and air or oxygen supplied to the cathode compartment.

### CATALYST COATED MEMBRANE (CCM):

A variety of techniques are known for CCM manufacture for applying an electrocatalyst coating composition similar to that described above onto the solid fluorinated polymer electrolyte membrane. Some known methods include spraying, painting, patch coating and screen, decal, pad or flexographic printing.

In one embodiment of the invention, the MEA (30), shown in FIG. 1, may be prepared by thermally consolidating the gas diffusion backing (GDB) with a CCM at a temperature of under 200° C., preferably 140-160° C. The CCM may be made of any type known in the art. In this embodiment, an MEA comprises a polymer electrolyte (SPE) membrane with a thin catalyst-binder layer disposed thereon. The catalyst may be supported (typically on carbon) or unsupported. In one method of preparation, a catalyst film is prepared as a decal by spreading the catalyst ink on a flat release substrate such as Kapton® polyimide film (available from the DuPont Company). After the ink dries, the decal is transferred to the surface of the SPE membrane by the application of pressure and heat, followed by removal of the release substrate to form a catalyst coated membrane (CCM) with a catalyst layer having a controlled thickness and catalyst distribution. Alternatively, the catalyst layer is applied directly to the membrane, such as by printing, e.g. by flexographic printing, and then the catalyst film is dried at a temperature no greater than 200° C.

### Membrane Electrode Assembly:

The CCM, thus formed, is then combined with a GDB to form the MEA (30). The MEA is formed, by layering the CCM and the GDB, followed by consolidating the entire structure in a single step by heating to a temperature no greater than 200° C., preferably in the range of 140-160° C., and applying pressure. Both sides of the MEA can be formed in the same manner and simultaneously. Also, the composition of the catalyst layer and GDB could be different on opposite sides of the membrane. Alternately, the membrane electrode assembly may be formed by placing a gas diffusion electrode adjacent each surface of the polymer electrolyte membrane. The gas diffusion electrode comprises a gas diffusion backing and an electrode prepared from an electrocatalyst containing composition. The electrocatalyst composition may comprise the homopolymers or copolymers of the invention as a binder in the composition.

The invention is illustrated in the following examples.

### EXAMPLES

### In-Plane Conductivity Measurement

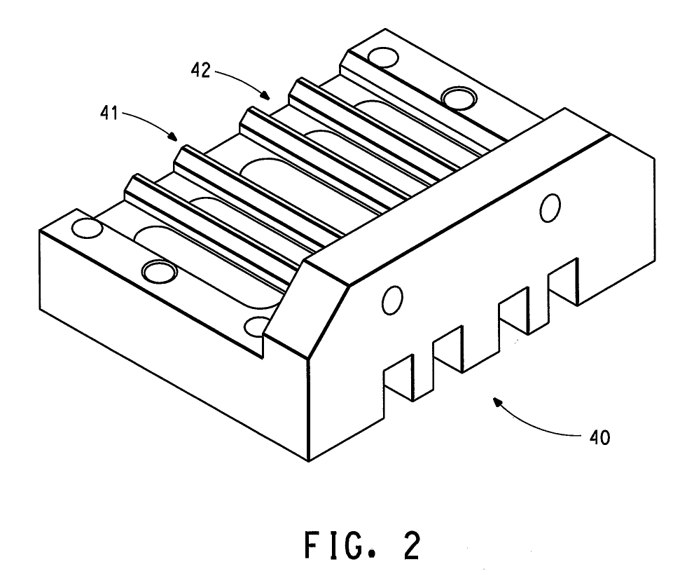

The in-plane conductivity of a membrane is measured under conditions of controlled relative humidity and temperature by a technique in which the current flows parallel to the plane of the membrane. A four-electrode technique is used similar to that described in an article entitled “Proton Conductivity of Nafion® 117 As Measured by a Four-Electrode AC Impedance Method” by Y. Sone et al., J. Electrochem. Soc., 143,1254 (1996) that is incorporated herein by reference. Referring to FIG. 2, a lower fixture (40) is machined from annealed glass-fiber reinforced PEEK to have four parallel ridges (41) containing grooves that support and hold four 0.25 mm diameter platinum wire electrodes. The distance between the two outer electrodes is 25 mm, while the distance between the two inner electrodes is 10 mm. A strip of membrane is cut to a width between 10 and 15 mm and a length sufficient to cover and extend slightly beyond the outer electrodes, and placed on top of the platinum electrodes. An upper fixture (not shown), which has ridges corresponding in position to those of the bottom fixture, is placed on top and the two fixtures are clamped together so as to push the membrane into contact with the platinum electrodes. The fixture containing the membrane is placed in a small pressure vessel (pressure filter housing), which is placed in a forced-convection thermostated oven for heating. The temperature within the vessel is measured by means of a thermocouple. Water is fed from a calibrated Waters 515 HPLC pump (Waters Corporation, Milford, Mass.) and combined with dry air fed from a calibrated mass flow controller (200 sccm maximum) to evaporate the water within a coil of 1.6 mm diameter stainless steel tubing inside the oven. The resulting humidified air is fed into the inlet of the pressure vessel. The total pressure within the vessel (100 to 345 kPa) is adjusted by means of a pressure-control let-down valve on the outlet and measured using a capacitance manometer (Model 280E, Setra Systems, Inc., Boxborough, Mass.). The relative humidity is calculated assuming ideal gas behavior using tables of the vapor pressure of liquid water as a function of temperature, the gas composition from the two flow rates, the vessel temperature, and the total pressure. Referring to FIG. 2, the slots (42) in the lower and upper parts of the fixture allow access of humidified air to the membrane for rapid equilibration with water vapor. Current is applied between the outer two electrodes while the resultant voltage is measured between the inner two electrodes. The real part of the AC impedance (resistance) between the inner two electrodes, R, is measured at a frequency of 1 kHz using a potentiostat/frequency response analyzer (PC4/750™ with EIS software, Gamry Instruments, Warminster, Pa.). The conductivity, κ, of the membrane is then calculated as

κ=1.00 cm/(R×t×w),

where t is the thickness of the membrane and w is its width (both in cm).

### Example 1

### Synthesis of BrC₆H₄SCF₂CF₂Br:

A 2 L flask was charged with 200.65 g (1.01 mol) of 4-Bromothiophenol and 600 mL of methanol. A solution of 77.6 g (1.18 mol) of potassium hydroxide in 200 mL of water was added via cannula to the stirred solution of 4-bromophenol over a period of 1.25 hours. The resulting solution was stirred for an additional four hours, then this solution was evaporated to dryness on a rotary evaporator and the resulting solid salt was dried at 140° C. and 0.1 mm Hg vacuum for four hours. The salt was ground and further dried at 140° C. and 0.1 mmHg vacuum for one additional hour. The dried salt was dissolved in 400 ml of DMSO under nitrogen and then transferred slowly into a dried flask containing 500 g (1.92 mol) of Br(CF₂)₂Br and 300 mL of DMSO at room temperature over 2.5 hrs. The resulting reaction mixture was stirred at 60° C. for 5 hours, at room temperature overnight, and then was diluted into 2-L of ice and water. The organic layer was separated and the aqueous solution was extracted with 3×100 mL of methylene chloride. The methylene chloride extracts were combined with the organic layer, washed with 3×200 mL water and dried over MgSO₄. The filtered CH₂Cl₂ product solution was concentrated on a rotary to obtain the crude product of 324.49 g (88.56% yield) as a brown liquid. ¹⁹F NMR: −62.8 (s, 2F), −85.6 (s, 2F).

### Example 2

### Preparation of p-BrC₆H₄SCF₂CF₂SO₂Na

A 1 L 3-necked flask fitted with a mechanical stirring device and a condenser was charged with 281.2 mL of water, 281 mL of DMF, 336.5 g of Na₂S₂O₄ (85% purity) and 169.4 g of NaHCO₃ under N₂. 324.5 g of BrC₆H₄SCF₂CF₂Br were added and the resulting mixture was stirred at 60° C. for 3.5 hours and then cooled to 5° C. After 1 L of ethyl acetate was added and the mixture stirred, the liquid layer was decanted and solids were washed with ethyl acetate (2×500 mL). The filtrates were combined with the decant and then the organic layer was separated, washed with saturated NaCl (2×250 mL), filtered and evaporated to give 329 g of yellow solids. ¹⁹F NMR (DMF-d₇): −86.0(s, 2F), −127.9 (s, 2F).

### Example 3

### Preparation of Br(C₆H₄)—S—CF₂CF₂SO₂Cl

A 3.0 L three necked round bottomed flask with cooling jacket was set up with a fritted sparge tube in a rubber septa, mechanical motor with glass shaft, bearing and Teflon® blade; and a ‘Y’ tube adapter with thermometer well and a dry ice condenser vented to a N₂ bubbler. The flask was cooled using a chilled re-circulation bath and charged with 760 mL deinized water, 585 mL of 1,1,2-trichlorotrifluoroethane (CFC-113), and 329.27 g. of Br(C₆H₄)—S—CF₂CF₂SO₂Na. The stirred reaction mixture was sparged with N₂ via the fritted glass tube for 30 minutes before 118 g of chlorine gas were added for 40 min at <10° C. After the addition was complete, the reaction mixture was allowed to warm to 20° C.; and transferred to a 3000 mL separatory funnel. The organic layer was separated, and the aqueous layer was washed with 2×100 mL CFC-113. CFC layers were washed with 2×200 mL of saturated NaCl solution. Combination of all organic layers was dried over MgSO₄; filtered and evaporated at 45-50° C. to give 263.9 g of a yellow hard waxy solid. ¹⁹F NMR(CDCl₃): −85.0 (s, 2F), −102.3 (s, 2F).

### Example 4

### Preparation of BrC₆H₄SCF₂CF₂SO₂F:

The oven dried flask was charged with 33.5 g (0.0864 mol) of p-Br-(C₆H₄)—S—CF₂CF₂SO₂Cl, 80 mL of dry acetonitrile and 7.92 g (0.136 mol) of KF under N₂ and the resulting mixture was stirred at 60° C. to 70° C. for 13 hrs. The reaction mixture was filtered and solids were washed with ether (2×100 mL). The combined filtrates were evaporated to give 30.53 g of residual liquid. ¹⁹F NMR(CDCl₃): +46.4 (s, 1F), −85.8 (s, 2F), −104.8 (s, 2F).

### Example 5

### Preparation of CF₂═CFC₆H₄SCF₂CF₂SO₂F:

A 1 L two necked flask fitted with rubber septa, a magnetic stirring bar, vented connector tube, and a dry ice condenser vented to a nitrogen purge tube bubbler was charged with 45 g (0.69 mol) of acid-washed Zn and 500-mL of DMF at room temperature. CF₂═CFBr was slowly added as a gas via the vented connector tube and allowed to condense at the dry ice to a suspension of Zn and DMF in the flask. After addition of 2 mL of bromine, an exothermic reaction occurred, and the mixture was stirred at room temperature for 2 hours, during which 99.1 g (0.616 mol) of CF₂═CFBr were added, and stirring was continued at 65° C. for 1.5 hours to give a CF₂═CFZnX solution.

An oven dried 250 mL two necked flask was charged with 2.80 g. of Pd(PPh₃)₄, 30 mL of dry DMF and 30.0 g of Br—(C₆H₄)—S—CF₂CF₂SO₂F under N₂. 142 mL of CF₂═CF—ZnX solution in DMF were transferred into the flask and the resulting reaction mixture was stirred at 70° C. for 7 hours and at room temperature overnight and then distilled into a dry ice cooled receiver at 71-72° C. and 0.35 mm Hg vacuum. The distillate was added to an equal volume of ice water and the organic layer was separated as the lower layer. The organic layer was washed with ice water twice and dried over MgSO₄. After removal of MgSO₄, liquid was distilled to give 0.636 g of bp 49-51° C./0.22 mmHg material and 21.7 g of product, bp 62-66° C./0.26 mmHg. ¹⁹F NMR: +44.2 (s, 1F), −81.5 (m, 2F), −98.9 (dd, J=71.6 Hz, J=37.7 Hz, 1H), −111.5 (s, 2F), −114.0 (dd, J=109.3 Hz, J=71.6 Hz, 1F), −117.3 (dd, J=71.6 Hz, J=37.7 Hz, 1F). −177.3 (dd, J=109.3 Hz, J=37.7 Hz, 1F). ¹H NMR: 7.50 (d, J=8.2 Hz, 2H), 7.32 (d, J=8.2 Hz, 2H).

### Example 6

### Preparation of CF₂BrCFBrC₆H₄SO₂CF₂CF₂SO₂F:

To a mixture of 30 g of CF₂═CFC₆H₄SCF₂CF₂SO₂F and 80 mL of CH₂Cl₂ was added 18 g of Br₂ slowly. After the addition was complete, the reaction mixture was stirred at room temperature for 3 hours and then evaporated to give a liquid residue, which was diluted with 130 mL of acetic acid. 30 g of CrO₃ were added to the solution, portion by portion, at room temperature (RT), and the resulting mixture was stirred at room temperature for 2 days. The reaction mixture was poured into 400 mL of ice water, filtered, and washed with water 3 times. The solid was dissolved in 150 mL of CH₂ Cl₂ and washed with water and brine, and dried over MgSO₄. After removal of CH₂Cl₂, residual solids were obtained. ¹⁹F NMR: 46.55(, 1F), −57.5 (dd, J=173.4 Hz, J=18.8 Hz, 1H), −58.6 (dd, J=170 Hz, J=15.1 Hz, 1F), −106.4 (s, 2F), −110.3 (s, 2F), −121.0 (m, 1F). ¹H NMR: 7.95 (d, J=8 Hz, 2H), 8.10 (d, J 8.0 Hz, 2H).

### Example 7

### Preparation of p-CF₂═CFC₆H₄SO₂CF₂CF₂SO₂F

To a stirred suspension of 28 g of Zn in 150 mL of ether was slowly added 30 g of CF₂BrCFBrC₆H₄SO₂CF₂CF₂SO₂F at room temperature. After 10 min, exothermic reaction occurred and the reaction mixture was stirred at 35° C. for 4 hrs and then filtered to remove solids, which were washed with ether. The filtrate was washed with 1% HCl solution, brine and dried over MgSO₄. After removal of the ether, 21.3 g of white solid product were obtained. +46.2 (s, 1F), −93.3 (dd, J=71.6 Hz, J=33.9 Hz, 1F), −106.2 (s, 2F), −107.5 (dd, J=107.5 Hz, J=71.6 Hz, 1F), −110.5 (m, 2F), −178.1 (dd, J=107.5 Hz, J=33.9 Hz, 2F). ¹H NMR: 8.12 (d, J=7.8 Hz, 2H), 7.83 (d, J=7.8 Hz, 2H).

### Example 8

### Homopolymerization of CF₂═CFC₆H₄SCF₂CF₂SO₂F Using Potassium Persulfate (KPS):

A 250 mL three necked round bottomed flask, fitted with a rubber septa, cold water condenser with a N₂ outlet/inlet and bubbler, a magnetic stirring bar, and a thermocouple, was charged with 50 mL of deionized water and 4.8 mL of 20 wt. % ammonium perfluorooctanoate (C8) aqueous solution. The solution was bubbled with N₂ for 30 minutes and then 11 g (30 mmol) of CF₂═CFC₆H₄SCF₂CF₂SO₂F were added to the flask via a syringe under N₂, followed by ultrasonication for 5 min. After heating to 50° C., 40 mg of potassium persulfate (KPS) in 2 mL of water were added and the resulting mixture was stirred at 50° C. 48 hours, and then an additional 40 mg of KPS in 2 mL of water were added. The mixture was continued to be stirred for 14 hours and then was frozen. After melting, the mixture was treated with 15 ml of 10% HNO₃ at 90° C. for 1.5 hours, then cooled to RT. The resulting solids were filtered and washed with water three times and further dried at 105° C. under vacuum/N₂ for 4 hours to give 9.9 g of polymer. The polymer was dissolved in 60 mL of THF, and then slowly poured into 400 mL of MeOH with stirring to precipitate the polymer, which was filtered, washed with MeOH and dried in a vacuum oven at 80° C. overnight to give 8.7 g of polymer. Polymer is soluble in THF, and GPC indicated Mw=2.06×10⁵ and Mn=4.98×10⁴.

### Example 9

### Preparation Membrane from Homopolymer:

An expanded PTFE film (48 mg) was immersed in 10% of homopolymer of CF₂═CFC₆H₄SCF₂CF₂SO₂F in THF at room temperature for 10 min. The film was removed and dried at 70° C. in a vacuum oven for 40 min. 127 mg of composite film were obtained. The film was immersed in 20% KOH in MeOH/water/DMSO (4/5/1 ratio) at 60° C. for 5 hrs. The film was removed, washed with water and immersed in 30 mL of 10% HNO₃ at 60° C. overnight and then washed with deionized water. After drying with a paper towel, 378 mg of film were obtained. Conductivity was measured at 120° C. at 25% to 95% relative humidity.

| Relative Humidity (RH) | Conductivity (mS/cm) |
|---|---|
| 25.1 | 25.5 |
| 50.1 | 58.5 |
| 95 | 215.0 |

1. A monomer comprising the structure 1a or 1b: 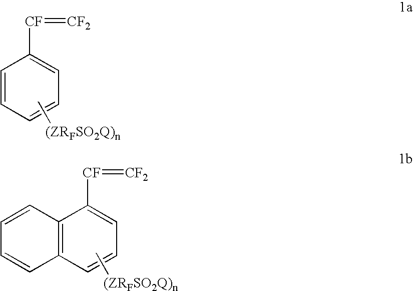
wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine; Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms, wherein the alkyl or aryl groups may be perfluorinated or partially fluorinated; and
n is 1 or 2 for 1a, and n is 1, 2, or 3 for 1b.

2. The monomer of claim 1 wherein R_{F} comprises (CF₂)_{q} wherein q=1 to 16, (CF₂)_{q}OCF₂CF₂ wherein q=1 to 12, or (CF₂CF(CF₃)O)_{q}CF₂CF₂ wherein q=1 to 6.

3. The monomer of claim 2 wherein R_{F} comprises (CF₂)_{q} wherein q=1 to 4; (CF₂)_{q}OCF₂CF₂ wherein q=1 to 4; or (CF₂CF(CF₃)O)_{q}CF₂CF₂ wherein q=1 to 2.

4. The monomer of claim 1 wherein n is 1 and Z is S or SO₂.

5. A homopolymer having the following structure 2a or 2b: 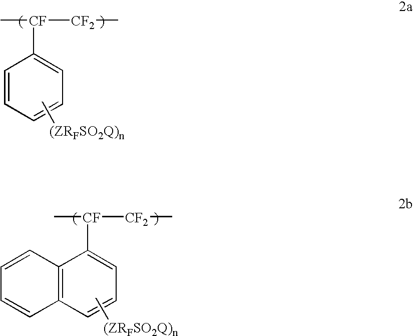
wherein Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and
n is 1 or 2 for 2a, and n is 1, 2, or 3 for 2b.

6. The homopolymer of claim 5 wherein R_{F} comprises (CF₂)_{q} wherein q=1 to 16, (CF₂)_{q}OCF₂CF₂ wherein q=1 to 12, or (CF₂CF(CF₃)O)_{q}CF₂CF₂ wherein q=1 to 6.

7. The homopolymer of claim 6 wherein comprises (CF₂)_{q} wherein q=1 to 4; (CF₂)_{q}OCF₂CF₂ wherein q=1 to 4; or (CF₂CF(CF₃)O)_{q}CF₂CF₂ wherein q=1 to 2.

8. The homopolymer of claim 5 wherein n is 1 and Z is S or SO₂.

9. A copolymer chosen from:
(a) copolymers comprising structure 3a or 3b: 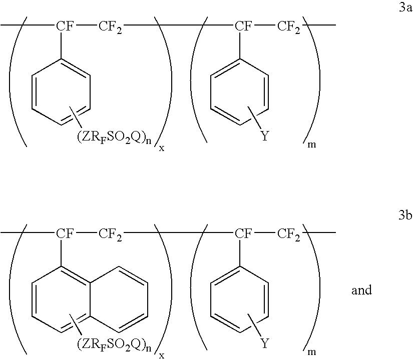
(b) cross linked copolymers comprising structure 4a or 4b: 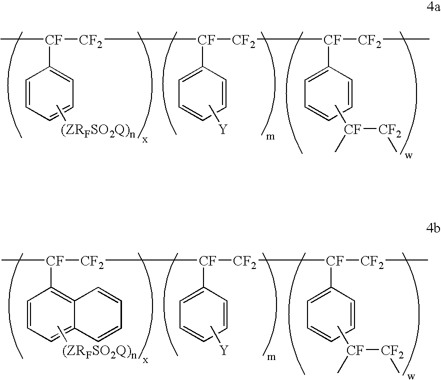
wherein in 3a, 3b, 4a or 4b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;
Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;
n is 1 or 2 for 3a and 4a, and n is 1, 2, or 3 for 3b or 4b; and
x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

10. The copolymer of claim 9 wherein R_{F} comprises (CF₂)_{q} wherein q=1 to 16, (CF₂)_{q}OCF₂CF₂ wherein q=1 to 12, or (CF₂CF(CF₃)O)_{q}CF₂CF₂ wherein q=1 to 6.

11. The copolymer of claim 10 wherein R_{F} comprises (CF₂)_{q} wherein q=1 to 4; (CF₂)_{q}OCF₂CF₂ wherein q=1 to 4; or (CF₂CF(CF₃)O)_{q}CF₂CF₂ wherein q=1 to 2.

12. The copolymer of claim 9 wherein Y comprises hydrogen, chlorine, fluorine, methyl, ethyl, methoxy, perfluoromethyl, perfluoroethyl, perfluorobutyl, perfluoromethoxy, and —CF₂CF(CF₃)OCF₂CF₃ and R²_{F} comprises methyl, ethyl, propyl, butyl, and phenyl, each of which may be partially fluorinated or perfluorinated.

13. The copolymer of claim 12 wherein R²_{F} is chosen from perfluoromethyl, perfluoroethyl, and perfluorophenyl.

14. The copolymer of claim 9 wherein n is 1 and Z is S or SO₂.

15. The copolymer of claim 9 wherein m, x and w are mole fractions, wherein m is 0.1 to 0.4; and x is 0.9 to 0.6 in structure 3a or 3b and wherein m is 0.2 to 0.6; x is 0.4 to 0.8; and w is 0.002 to 0.01 in structure 4a or 4b.

16. A polymer electrolyte membrane prepared from
(a) a homopolymer or copolymer chosen from: 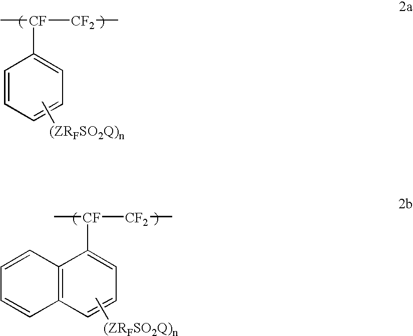
wherein in 2a or 2b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroaikene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and
n is 1 or 2 for 2a, and n is 1, 2, or 3 for 2b;
(b) copolymers comprising structure 3a or 3b: 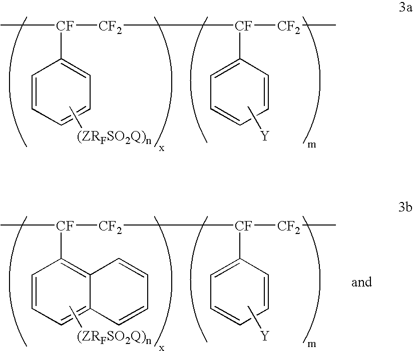
(c) cross linked copolymers comprising structure 4a or 4b: 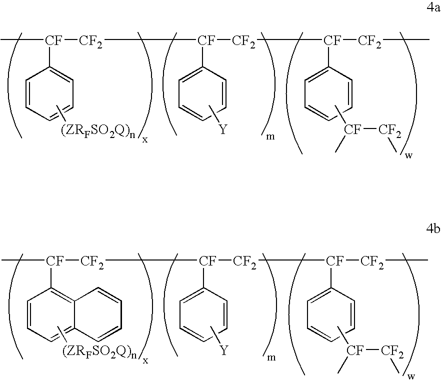
wherein in 3a, 3b, 4a or 4b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;
Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;
n is 1 or 2 for 3a and 4a, and n is 1, 2, or 3 for 3b or 4b; and
x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

17. The polymer electrolyte membrane of claim 16 further comprising a porous support.

18. (canceled)

19. (canceled)

20. (canceled)

21. (canceled)

22. (canceled)

23. (canceled)

24. A polymer electrolyte membrane chosen from:
(a) a membrane comprising the chemical structure: 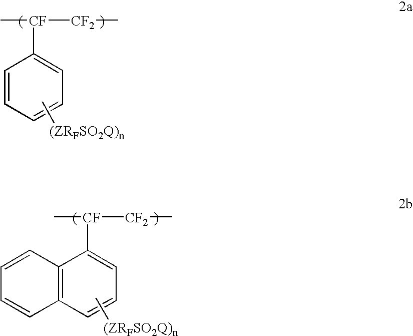
wherein in 2a or 2b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and
n is 1 or 2 for 2a, and n is 1, 2, or 3 for 2b;
(b) a membrane comprising the structure 3a or 3b: 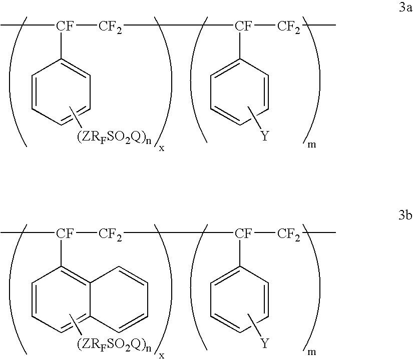
and (c) a membrane comprising the structure 4a or 4b: 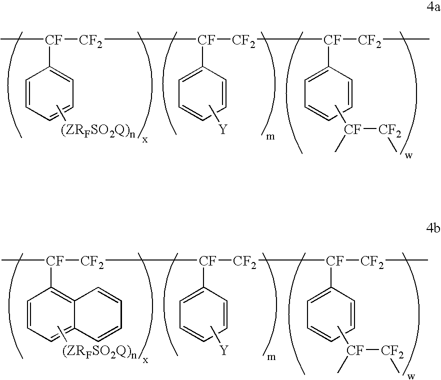
wherein in 3a, 3b, 4a or 4b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH2, —N(M)SO2R2F, and —C(M)(SO2R2F)2, wherein M comprises H, an alkali cation, or ammonium, and R2F groups comprise alkyl of 1 to 14 carbon atoms that may optionally include ether oxygens or aryl of 6 to 12 carbon atoms where the alkyl or aryl groups may be perfluorinated or partially fluorinated;
Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;
n is 1 or 2 for 3a and 4a, and n is 1, 2, or 3 for 3b or 4b; and x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1

25. A membrane electrode assembly comprising a polymer electrolyte membrane, having a first surface and a second surface, wherein the membrane is prepared from a homopolymer or copolymer chosen from:
(a) a homopolymer comprising structure 2a or 2b: 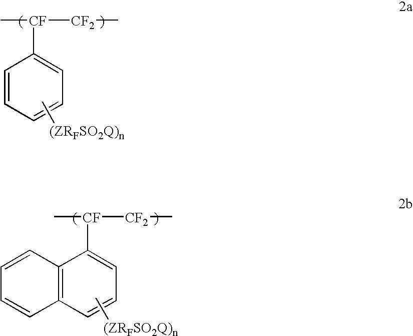
wherein in 2a or 2b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and
n is 1 or 2 for 2a, and n is 1, 2, or 3 for 2b
(b) copolymers comprising structure 3a or 3b: 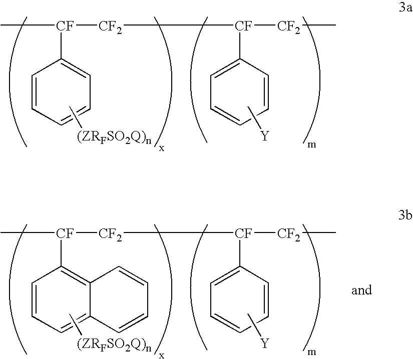
(c) cross linked copolymers comprising structure 4a or 4b: 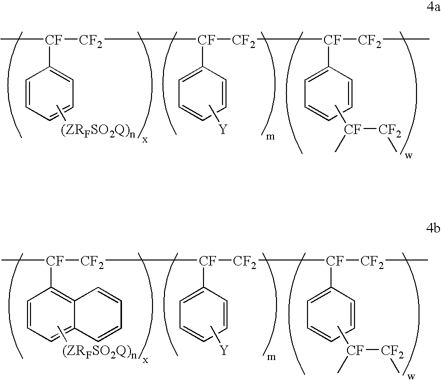
wherein in 3a, 3b, 4a or 4b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms where the alkyl or aryl groups may be perfluorinated or partially fluorinated;
Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;
n is 1 or 2 for 3a and 4a, and n is 1, 2, or 3 for 3b or 4b; and
x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

26. The membrane electrode assembly of claim 25 wherein the polymer electrolyte membrane further comprises a porous support.

27. (canceled)

28. (canceled)

29. (canceled)

30. (canceled)

31. (canceled)

32. (canceled)

33. (canceled)

34. (canceled)

35. (canceled)

36. An electrochemical cell comprising a membrane electrode assembly, wherein the membrane electrode assembly comprises a polymer electrolyte membrane, having a first surface and a second surface, wherein the membrane is prepared from a homopolymer or copolymer chosen from:
(a) a homopolymer comprising the structure 2a or 2b: 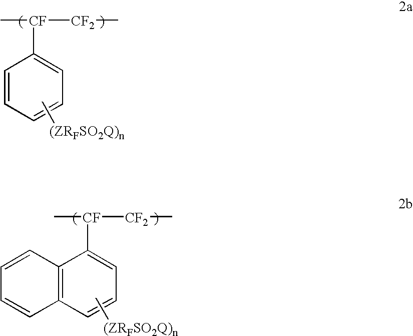
wherein in 2a or 2b:
Z comprises S, SO₂, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
R_{F} comprises a linear or branched perfluoroalkene group of 1 to 20 carbon atoms, optionally containing oxygen or chlorine;
Q is chosen from F, —OM, —NH₂, —N(M)SO₂R²_{F}, and —C(M)(SO₂R²_{F})₂, wherein M comprises H, an alkali cation, or ammonium, and R²_{F} groups comprise alkyl of 1 to 14 carbon atoms that may optionally include ether oxygens or aryl of 6 to 12 carbon atoms wherein the alkyl or aryl groups may be perfluorinated or partially fluorinated; and
n is 1 or 2 for 2a, and n is 1, 2, or 3 for 2b
(b) copolymers comprising structure 3a or 3b: 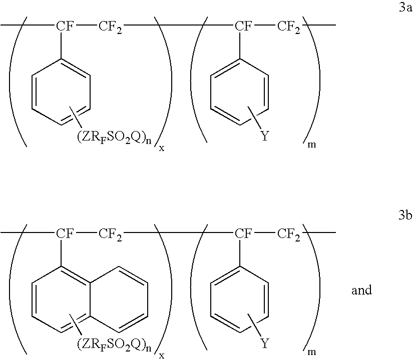
(c) cross linked copolymers comprising structure 4a or 4b: 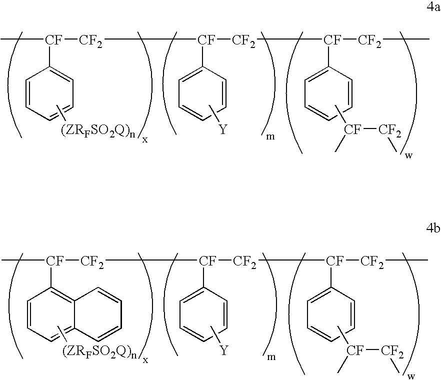
wherein in 3a, 3b, 4a or 4b:
Z comprises S, SO2, or POR wherein R comprises a linear or branched perfluoroalkyl group of 1 to 14 carbon atoms optionally containing oxygen or chlorine, an alkyl of 1 to 8 carbon atoms, an aryl group of 6 to 12 carbon atoms or a substituted aryl group of 6 to 12 carbon atoms;
RF comprises a linear or branched perfluoroalkene group of 1 to 20 carbon atoms, optionally containing oxygen or chlorine;
Q is chosen from F, —OM, —NH2, —N(M)SO2R2F, and —C(M)(SO2R2F)2, wherein M comprises H, an alkali cation, or ammonium, and R2F groups comprise alkyl of 1 to 14 carbon atoms that may optionally include ether oxygens or aryl of 6 to 12 carbon atoms where the alkyl or aryl groups may be perfluorinated or partially fluorinated;
Y comprises H; halogen such as Cl, Br, F or I; linear or branched alkyl or perfluoroalkyl groups, wherein the alkyl group comprises C1 to C10 carbon atoms; or a perfluoroalkyl group containing oxygen, chlorine or bromine, and wherein the alkyl group comprises C1 to C10 carbon atoms;
n is 1 or 2 for 3a and 4a, and n is 1, 2, or 3 for 3b or 4b; and
x, m, and w are mole fractions wherein x is 0.99 to 0.05; for 3a and 3b m is 0.01 to 0.95 and x+m=1; for 4a and 4b m=0 to 0.95, w=0.0001 to 0.10 x+m+w=1.

37. The electrochemical cell of claim 36 wherein the electrochemical cell is a fuel cell.

38. The fuel cell of claim 37 wherein the polymer electrolyte membrane further comprises a porous support.

39. (canceled)

40. (canceled)

41. (canceled)

42. (canceled)

43. (canceled)

44. The fuel cell of claim 37 wherein the fuel is an alcohol or ether.

45. The fuel cell of claim 37 wherein the fuel is methanol or hydrogen.

46. (canceled)
